(19) United States
(12) Patent Application Publication
(10) Pub. No.: US 2024/0312610 A1
(43) Pub. Date: Sep. 19, 2024

(54) VIRTUAL EYE DISEASE PROGRESSION MONITORING AND DIAGNOSTICS

(71) Applicant: Digiteyez Corporation, WASHINGTON, DC (US)

(72) Inventors: Carole Egerton, Hitchin (GB); Ali Hashim, Seattle, WA (US); Brandon Zimmerman, Washington, DC (US)

(73) Assignee: Digiteyez Corporation, WASHINGTON, DC (US)

(21) Appl. No.: 18/607,069

(22) Filed: Mar. 15, 2024

Related U.S. Application Data
(60) Provisional application No. 63/452,378, filed on Mar. 15, 2023.

(51) Int. Cl. G16H 40/20; G16H 40/67

(52) U.S. Cl. CPC G16H 40/20; G16H 40/67

(57) ABSTRACT

A system and method includes managing eye disease progression using a story-led monthly eye test that monitors the disease in patients, particularly in pediatric patients aged 7-14 years old. Patients are invited to take the eye test monthly via the story-led, gamified solution. Administrators of the account have access to the results of the tests and further monitor the test taking of the patients. Eye care specialists have access to a platform for tracking the monthly test results and further monitors the disease progression.

### CROSS-REFERENCE TO RELATED APPLICATIONS

This applications claims priority to U.S. Provisional Application No. 63/452,378, filed Mar. 15, 2023, the contents of which are hereby incorporated by reference in their entirety.

### FIELD OF THE DISCLOSURE

The present disclosure is directed to a system and method for managing eye disease progression using a story-led monthly eye test that monitors the disease in patients, particularly in pediatric patients. The children and their parents are referred to the product via their optometrist who has diagnosed them, for example during the early stages of the eye disease and recommends tracking the progression of the eye disease.

### BACKGROUND OF THE DISCLOSURE

Myopia, also known as near sightedness, is a common refractive error where distant objects appear out of focus. Nearsightedness occurs either because the eyeball is too long, or because the eye's refractive power is too strong due to the shape of the cornea (lens) inside the eye. Myopia can be diagnosed during a general eye exam by a visual acuity test to measure vision at distances, refraction test to determine the correct prescription for glasses and/or slit-lamp exam to assess the structure of the eyes. A visual acuity test, for example, checks how sharp your vision is at a distance. The patient covers one eye, and in response to instructions from the eye care specialist, reads an eye chart with different sized letters or symbols. The same process is carried out for the other eye. In children, special charts are designed and used.

The AAP (American Academy of Pediatrics), AAPOS (American Association for Pediatric Ophthalmology and Strabismus) and AAO (American Academy of Opthalmology) recommend screening for eye disease in children as early as one year old, with visual acuity testing generally attempted at age four. Detecting eye disease in patients, particularly in young children, can be tough and there is a need for an accurate and seamless process for the patients. What is needed is clinical quality results providing accurate test results along with a better experience for the patient. Additionally, a process which allows the users to use their own and familiar devices can provide greater patient satisfaction. Current home vision tests for children and adults can be rudimentary, with the results, generally being inaccessible to the eye care specialist. Therefore, there is a need for tracking monthly test results and access to measuring disease progression.

A method for measuring the progression of an eye disease through monthly tracking of test results to understand the diagnosis of eye diseases is needed.

### SUMMARY

The present disclosure is directed to a system and method for diagnosing eye diseases and providing a platform for eye specialists to communicate with patients. A story-led monthly eye test monitors eye diseases in patients, in particular, pediatric patients. This story-led method and system may be implemented to encompass additional diagnostic capabilities, physical products such as glasses and contact lenses, books, films, stand-alone games and additional merchandise or content.

Visual functions have a tremendous impact on any patient's wellbeing. The systems and methods described herein allow patients to take eye tests on a monthly basis via a story-led, gamified solution. As vision plays a critical role in a person's life, diagnosing eye diseases is an important factor in ensuring a person's health. Eye diseases, for example, myopia especially in children can be hard to diagnose as they would have a difficult time recognizing and communicating that their vision may be impaired. Undiagnosed eye conditions can hinder development in children and impact day to day life of both children and adults. Therefore, it is important to have access to effective and engaging forms of testing for eye diseases.

The system and method described herein allow patients to access tests through their own devices, providing engaging and reliable methods for taking the test which are then accessed by eye care professionals to assess. Eye care professionals access a portal to track patients' monthly test results and will be alerted when disease progression goes outside of set threshold parameters. The system, through the portal, will allow eye care professionals to communicate with patients and schedule video or in-person appointments. Further, the system and methods described herein include a digital method of extrapolating user's acuity through a measured contrast sensitivity function.

The system and method described herein may direct the child, or other user, to perform certain tasks interacting with a visual display so that the system can obtain data and assess user vision. For example, the system may include interfaces displayed that test vision acuity (such as the LEA vision test), contrast perception, and astigmatism. For example, for visual acuity the system may include an interface that the user interacts with when standing a certain distance away. The interface may display one or more shapes or symbols, and ask the user to identify the shapes or symbols as the sizes change. The system can record responds by capturing verbal answers from the users received from a microphone, or the system may include an input for the user to enter answers such as a touchscreen, remote device, or mouse. The system may, for example, shrink the size of the shapes or symbols until the user can no longer correctly identify them. This can be performed for the left and right eyes individually and both eyes together. The system records the user responses for the different sizes of the shapes or symbols, and uses this to determine a vision acuity of the user.

The system may also test for contrast detection by providing a contrast wheel on either the left or side of the user interface against a background of similar color to the contrast wheel. The user may be asked to identify which side of the interface the contrast image is located on. When the user is correct, the contrast of the contrast wheel image may be decreased so it is closer to the background color. The test continues until the user is unable to detect which side of the interface the contrast image is located on. This can be performed for the left and right eyes individually and both eyes together. The responses, which may verbal or input through a user input interface such as a touchscreen, mouse, or remote input device, are recorded and used to determine contrast detection of the user.

The system may further include interfaces to test astigmatism. The user may be shown images on an interface that include series of lines. The user may be asked to cover one eye, and to identify any lines that appear darker by selecting such lines. The selection may be done through a touchscreen, mouse, or other user input device. The user responses are recorded, and used by the system to test for astigmatism.

The system may display these interfaces for the different tests as part of an interactive story or game, as explained above. The user may receive points or other items within the game based on completion of the tasks. The system may further record user responses to the tests over time, with the user interacting with the game and interfaces at different intervals. Thus, the system can obtain information allowing eye care professionals to monitor a user's vision over time. The responses recorded by the system described herein may be provided to an eye care provider, such as an optometrist, who can analyze the results, monitor progression, and make recommendations to the patient.

### BRIEF DESCRIPTION OF THE DRAWINGS

The present technology will be better understood on reading the following detailed description of non-limiting embodiments thereof, and on examining the accompanying drawings, in which:

FIG. 1A is a flow chart showing steps in a method of using the system and interfaces described herein for managing eye disease data.

FIG. 1B is a high-level diagram showing the components of a computer system for the story-led monthly eye test.

FIG. 2 provides an example for a landing page which prompts the user to either login or sign up.

FIG. 3 provides a screen shot asking a user to answer questions to determine if the patient is to be excluded from taking the test.

FIG. 4 is an example of a page to launch the game, including providing instruction for setting up for the test.

FIG. 5A provides an illustration of an example interface for use in a test of the visual acuity of the user.

FIG. 5B provides an illustration of an example interface for use in a test of the visual acuity of the user, showing a shape or symbol to be identified.

FIG. 6 provides an example of a visual contrast test interface.

FIG. 7 provides an example of an interface, and images, that may be used as part of an astigmatism test presented to the user during the game.

FIG. 8 provides an example of the conclusion of the test and story, followed with displaying of a countdown timer showing when the test and story can be accessed next time.

FIG. 9 provides an example of an account page for managing user profiles and health information.

FIG. 10 displays a portal for the provider for tracking health information after each test is taken.

### DETAILED DESCRIPTION

The foregoing aspects, features, and advantages of the present disclosure will be further appreciated when considered with reference to the following description of embodiments and accompanying drawings. In describing the embodiments of the disclosure illustrated in the appended drawings, specific terminology will be used for the sake of clarity. However, the disclosure is not intended to be limited to the specific terms used, and it is to be understood that each specific term includes equivalents that operate in a similar manner to accomplish a similar purpose. Additionally, like reference numerals may be used for like components, but such use should not be interpreted as limiting the disclosure.

When introducing elements of various embodiments of the present disclosure, the articles “a”, “an”, “the”, and “said” are intended to mean that there are one or more of the elements. The terms “comprising”, “including”, and “having” are intended to be inclusive and mean that there may be additional elements other than the listed elements. Any examples of operating parameters and/or environmental conditions are not exclusive of other parameters/conditions of the disclosed embodiments. Additionally, it should be understood that references to “one embodiment”, “an embodiment”, “certain embodiments”, or “other embodiments” of the present disclosure are not intended to be interpreted as excluding the existence of additional embodiments that also incorporate the recited features. Furthermore, reference to terms such as “above”, “below”, “upper”, “lower”, “side”, “front”, “back”, or other terms regarding orientation or direction are made with reference to the illustrated embodiments and are not intended to be limiting or exclude other orientations or directions. Like numbers may be used to refer to like elements throughout, but it should be appreciated that using like numbers is for convenience and clarity and not intended to limit embodiments of the present disclosure. Moreover, references to “substantially” or “approximately” or “about” may refer to differences within ranges of +/−10 percent.

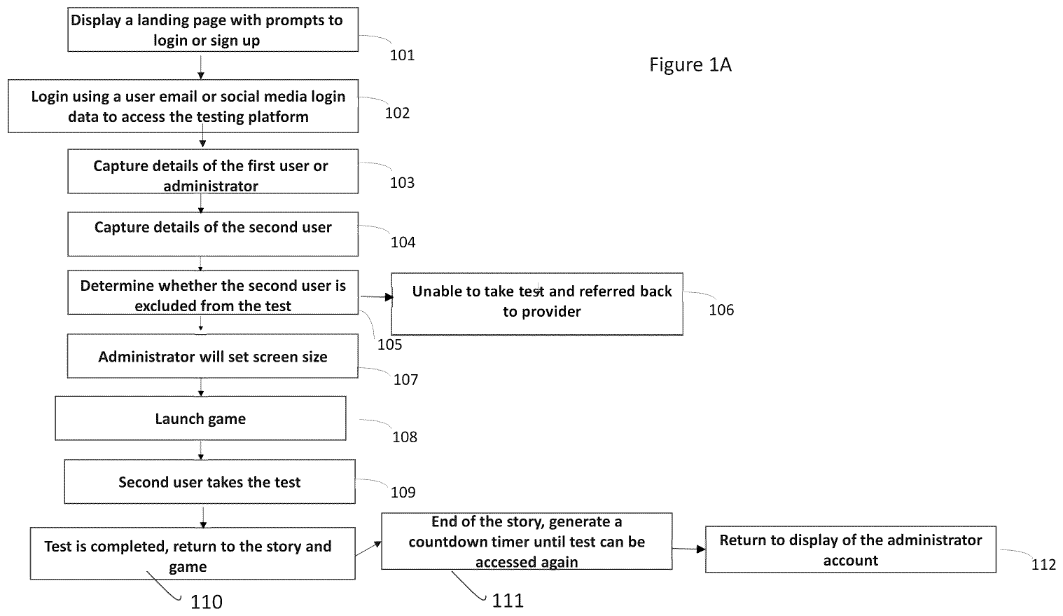

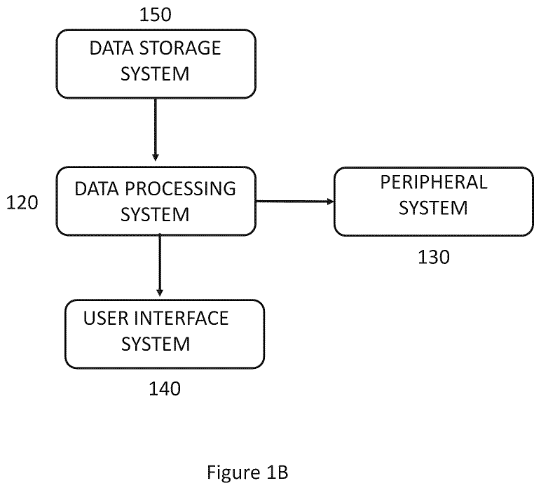

The present disclosure is directed to a system and method for eye disease management through a story-led monthly eye test which monitors eye disease in patients, in particular, pediatric patients. A platform is accessed by users being led through a story including testing components, in which a user performs a series of steps interacting with platform and user interfaces thereof to conduct vision tests. A representation of this overall process is illustrated in FIG. 1A. As shown in FIG. 1A, the process starts with the user logging in to the system and receiving instructions for conducting the eye test. The system will then capture information about the user, determine parameters of the user device such as screen size, and launch the game. The user then interacts with the game through a series of interfaces presented on the user device, during which vision tests are conducted and user responses to such tests recorded.

FIG. 1B is a high-level diagram showing the components of a computer system for accessing a story-led monthly eye test that monitors eye disease in patients. The system includes a data processing system 120, a peripheral system 130, a user interface system 140, and a data storage system 150. The peripheral system 130, the user interface system 140 and the data storage system 150 are communicatively connected to the data processing system 120. These systems may be included within a desktop computer, or within a mobile device, such as a smartphone, tablet, or PDA. Alternatively, the patient data may be transmitted to a separate system for processing. For example, the desktop computer may transmit data to a server on a cloud computing network. The server may process the data, and transmit back a visual simulation and an invitation to schedule video or in-person appointments.

The data processing system 120 includes one or more data processing devices that implement the processes of the various embodiments of the present disclosure, including the example processes described herein. The data processing devices may be, for example, a central processing unit (“CPU”), a desktop computer, a laptop computer, a mainframe computer, a personal digital assistant, a smartphone, a tablet, a digital camera, cellular phone, or any other device for processing data, managing data, or handling data.

The data storage system 150 includes one or more processor-accessible memories configured to store information, including software instructions executed by the processor and captured image data. The data storage system 150 may be a distributed processor-accessible memory system including multiple processor-accessible memories communicatively connected to the data processing system 120 via a plurality of computers or devices. On the other hand, the data storage system 150 need not be a distributed processor-accessible memory system and, consequently, may include one or more processor-accessible memories located within a single data processor or device. The processor-accessible memory may be any processor-accessible data storage device, whether volatile or nonvolatile, electronic, magnetic, optical, or otherwise, including but not limited to, registers, floppy disks, hard disks, Compact Discs, DVDs, flash memories, ROMs, and RAMs.

The system components may be communicatively connected in any manner that enables transmissions of data between components, including wired or wireless transmissions between devices, data processors, or programs in which data may be communicated. This connection may include a connection between devices or programs within a single data processor, a connection between devices or programs located in different data processors, and a connection between devices not located in data processors at all. In this regard, although the data storage system 150 is shown separately from the data processing system 120, the data storage system 150 may be stored completely or partially within the data processing system 120. Further in this regard, although the peripheral system 130 and the user interface system 140 are shown separately from the data processing system 120, one or both of such systems may be stored completely or partially within the data processing system 120.

The peripheral system 130 may include one or more devices configured to provide patient data records to the data processing system 120. For example, the peripheral system 130 may include cellular phones, or other data processors. The data processing system 120, upon receipt of patient data records from a device in the peripheral system 130, may store such patient data records in the data storage system 150. The peripheral system 130 does not need to be external to the device that includes the data processing system 120, user interface system 140, and data storage system 150.

The user interface system 140 may include a touch screen, touch pad, keypad, mouse, keyboard, another computer, or any device or combination of devices from which data is input to the data processing system 120. In this regard, and as noted above, although the peripheral system 130 is shown separately from the user interface system 140, the peripheral system 130 may be included as part of the user interface system 140. The user interface system 140 also may include a display device, a processor-accessible memory, or any device or combination of devices to which data is output by the data processing system 120. In this regard, if the user interface system 140 includes a processor-accessible memory, such memory may be part of the data storage system 150 even though the user interface system 140 and the data storage system 150 are shown separately in FIG. 1B.

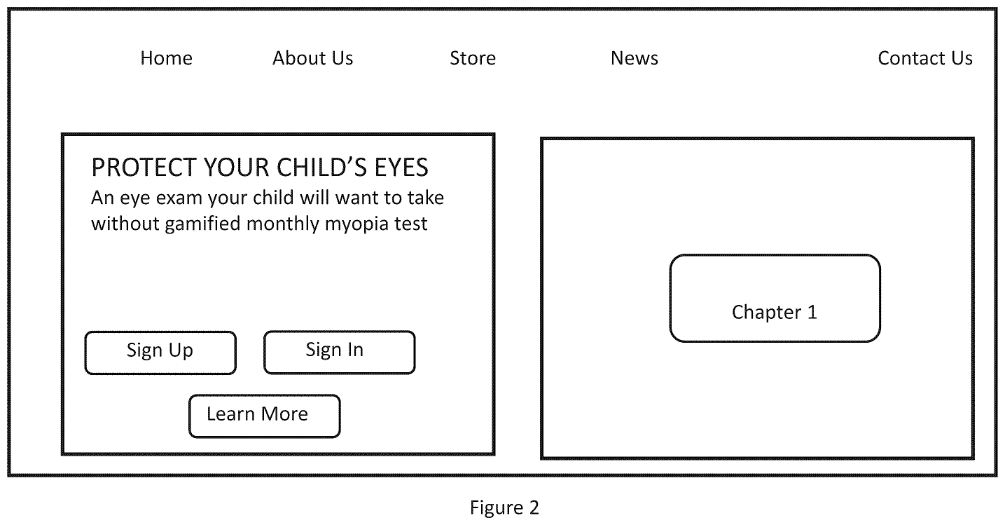

Referring to FIG. 1A, a method for a story-led monthly eye test monitoring eye disease in patients include the following steps. First, a landing page is displayed prompting the user to login or sign up to access the platform (step 101). The administrator, generally the parent of a pediatric patient, would access this landing page in response to an instruction from an eye care provider or professional. The landing page, depicted in FIG. 2 indicates the chapter being accessed with reference to the story and game to lead the user through the eye test. For example, the story depicted “Bo & Nomi” serve to provide an engaging narration while collecting eye health information for providing a proactive interaction between patient and eye care professional. In most cases, the user first accessing the landing page would be a parent looking to have their child tested for eye disease and further monitoring the progression of and diagnosing eye diseases. The parent or administrator would access the landing page based on a referral provided by the eye care professional directing the patients, parents or administrators to monitor a diagnosis of an eye disease, such as Myopia.

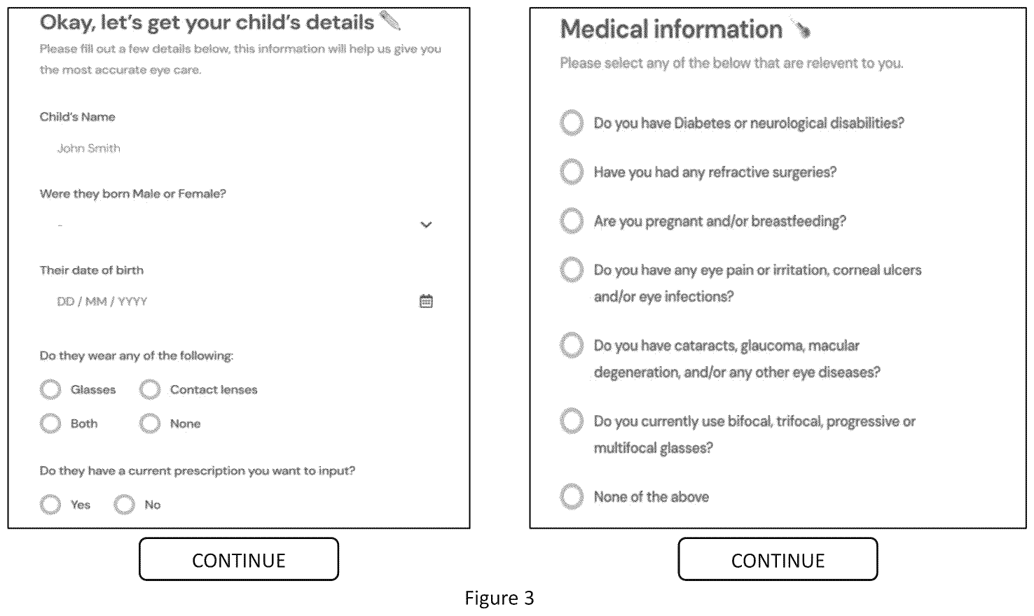

Next, referring back to FIG. 1A, the user, parent or administrator logs in to the platform using a user email or social media login information (step 102). The user captures the details of the first user, such as an administrator or parent (step 103), with a following page that captures the patient, such as a pediatric patient's information (step 104). The platform further determines if the patient is able to or qualified to take the test (step 105). Medical information is collected based on queries related to the patient's conditions, further determining whether the patient is excluded from taking the test. In most cases, a parent or administrator would answer the questions presented on behalf of a pediatric patient to determine if the child qualifies for the test. Based on the answers to the questions presented, for example, if the parent answers yes to any of the questions, the process for testing would be unable to continue and the administrator would be referred back to consult with the eye care professional (step 106). Examples of the questions that may be presented, and the interfaces on which they may be presented, are illustrated in FIG. 3.

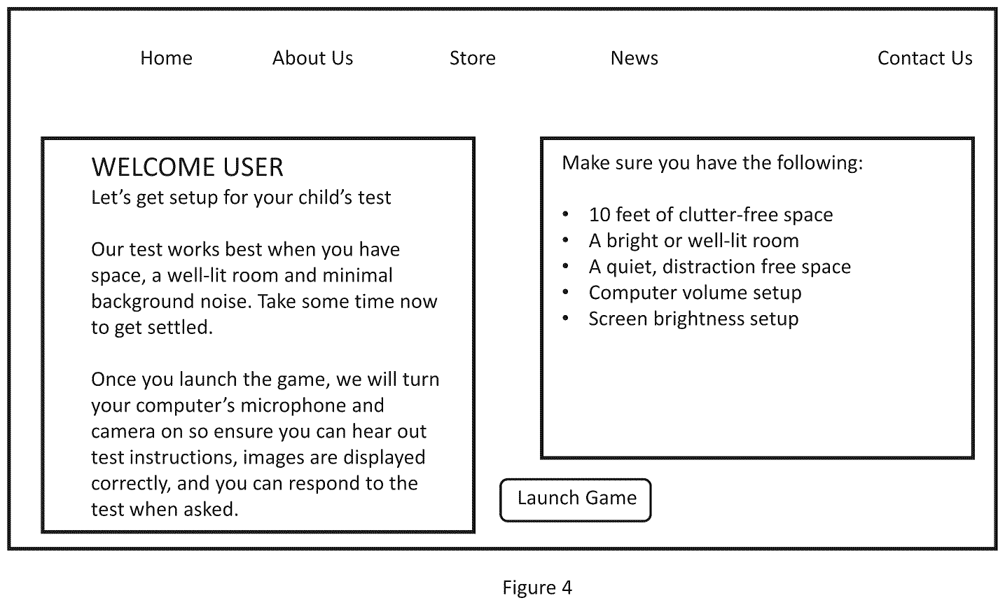

Step 107 of FIG. 1A launches the game including setup instruction for the patient's test. For example, the instructions for the eye test depicted instruct the user to evaluate the environment in which the test is to take place. The instructions are intended for the parent or administrator to get the patient suitably set up to take the test. The administrator and parent further sets up the screen size to ensure that the images are displayed at the correct size (step 107). A slider is provided to adjust the display size appropriate for taking the test. The parent or administrator upon carrying out the instructions, then hands over the device and test to the child to launch the game leading to the test (step 108). For example, an interface such as that shown in FIG. 4 may be presented with instructions to the user on how to set up the test. The user may be instructed to make sure that there is at least 10 feet of clear space, that the room is sufficiently lit, that the computer volume and microphone are working, and that the screen brightness is set to a sufficient level. The system may include using the video camera of the user device to capture an image of the user, such as the child, taking the test. The system may take an image of the child's eyes as they move away from the device, in order to capture pupillary distance. The device may be moved until the system determines that the eyes of the child are at the appropriate position. When the system determines that the child is in the correct position, it may produce an indication that position is correct and testing may begin. This may be, for example, an audio alert or tone, a visual indication, such as a green border or box on the screen, or a combination of audible and visual alerts. After one user completes the test, the system and platform may allow for a second user to log in an also complete the testing, as shown at step 109 in FIG. 1A.

Taking the test by the patient, begins with an introduction to a story or narration, generally starting with a first chapter. The narration introduces the characters to the user participating in the test, such as a child. The test taker traverses a series of screens introducing both the elements of the story, including characters and game mechanisms. Game mechanisms include items such as, crystals stored as part of the game and story. Engaging the patient in the story includes conveying a narration and further asking the patient to participate in the story with games and finding solutions to the character's queries. For example, Bo asks the patient to help her find symbols, leading the patient to take the eye test. The components of the story, game and the eye test are combined where the patient in participating in the game and story is able to conduct and provide results for their eye test. The patient is provided instructions for taking the test, for example, providing instructions of symbols to look for.

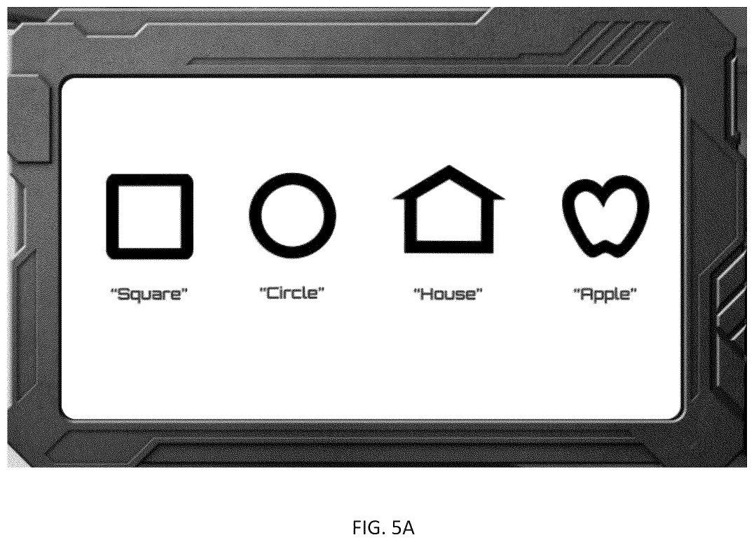

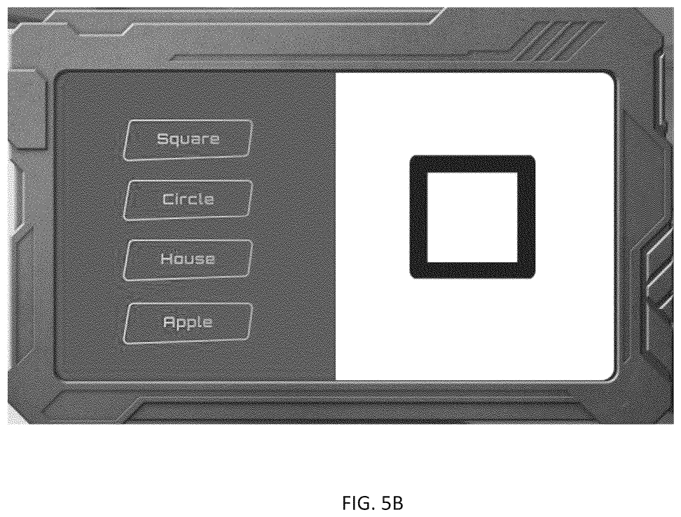

For example, the system may include interfaces to test the user's visual acuity. This may include interfaces to conduct an LEA vision test using symbols or shapes. Letters or other visual indicators may also be used on the interface to conduct the test. As shown in FIG. 5A, for example, the system may display a plurality of symbols to the user, and indicate to the user that they are indicate which of the plurality of symbols they see during the test. The symbols depicted can include square, circle, house, apple or any form of an image or symbol depicted to the patient for testing their visual functions. Providing instructions further includes calibrating, through accepting of user input confirming the patient's understanding of the symbols depicted. In response to the patient correctly responding to the queries, the narration and characters further confirm the answers. The patient is awarded as the respond to the queries correctly, further engaging and setting up the patient to prepare for the eye test. The instructions include adjusting the position of the patient with reference to the device displaying the test data. For example, the patient should maintain a distance of ten feet for taking the eye test. The narration further indicates that the platform accepts voice input and is voice activated. The patient is further instructed to test and input a voice command. The system confirms the quality of the voice input and moves forward with the narration, preparing for the test.

The system also captures the image of the child's eyes, including scanning the yes to determine pupillary distance. Instructions are provided to the patient to get into position, including placing the client device on a table and ensuring that space is provided to maintain the distance between the patient and the device. When the patient is ready to move away from the screen, the screen begins to adjust, capturing the patient's position with reference to the client device. The screen shows image of patient as they move backwards into the correct position. The screen further provides a visual indication when the patient has reached the correct position from which to take the eye test. The test begins with display of a symbol or image, with the patient asked to identify different symbols or images on the screen. For example, as shown in FIG. 5B, the user may be shown an interface with a picture of a square, along with prompts asking the user whether they see a square, circle, house, or apple. The images and prompts are demonstrative, and any images or prompts may be used. The user may give an answer by indicating which image the see. This can be recorded by voice recognition, with the user verbally indicating which image they see. The answer may also be recorded through a user input interface, such as a touchscreen or mouse. A parent or other person working with the child could also, for example, select the prompt on the interface, with the interface being for example a touch screen interface, to indicate the selection made by the user. The size of the symbols or images are modified (e.g. decreased) when the patient is able to provide a correct answer with the process continuing until the patient is unable to provide an appropriate response (e.g. can no longer identify the image or symbol). Each of the tests depicted in the games are generally performed for both eyes, left eye and right eye individually. The patients answers are recorded by the system, such that they can be analyzed and sent to an eye care provider, such as an optometrist.

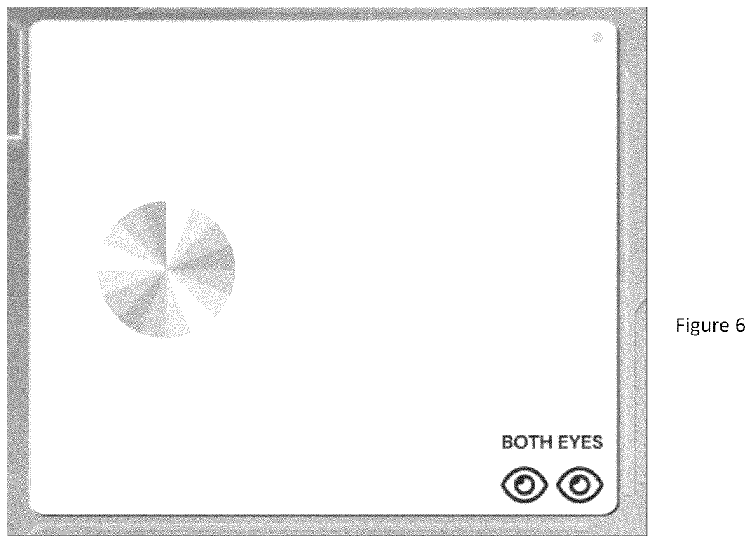

The system and platform may further include interfaces to test for patient contrast sensitivity, where patient contrast sensitivity is measured to determine visual functions. For example, a patient may be shown a contrast image (FIG. 6) on one side of the screen (e.g. left or right hand side) and asked to provide feedback concerning the image, including identifying colors of the image and/or determining the side of the screen the patient sees the image. If the patient identifies the correct or appropriate response, the image is further modified, including modifying the contrast of the image. After the contrast image is modified, the patient may again be asked to determine the colors of the image or the side of the screen they see the image. Identifying the correct or appropriate response includes, correctly identifying the colors of the image and/or the position of the image on the screen. Modifications to the image include changes in contrast and changes in the position of the image with reference to the screen. This process is carried out for both eyes, left eye and right eye. While a white backgrounds is shown in FIG. 6 for illustrative purposes, the test could be conducted with a black background, or a background of any other color, in order to test the patient's contrast sensitivity.

For example, in an embodiment, a system and method may measure the contrast acuity of a patient. The system and method may be implemented into a digital platform that employs a test for extrapolating a patient's contrast acuity to determine the patient's measured contrast sensitivity threshold. The patient's contrast sensitivity threshold is first determined upon one or more responses from the patient that a particular contrast image, such as the contrast image shown in FIG. 6, is “Not Clear.” When the responses during this test are normalized against various visual acuity models, the numerical output of the normalization is the patient's resulting contrast acuity. As contrast acuity decreases, a limit is reached in which a patient can no longer distinguish shades of a color in the contrast image. The limit that is reached is the patient's contrast sensitivity threshold. In practice, the contrast sensitivity threshold represents the minimal object size that a user can see under a given lighting condition. It is analogous to how a patient's visual acuity varies with room brightness, which makes it a powerful way of assessing the visual performance of the patient's eye.

A method for determining the contract threshold of the patient's eye may include the use of the Rayleigh Criterion. The Rayleigh criterion mathematically represents the smallest size an optical instrument can distinguish, represented by the formula: x=(1.22*λd)/D. In the formula, x is the pitch distance between adjacent contrast limbs, λ is the wavelength of light, d, is the distance between the user and the contrast optotype, and D is the lens diameter of the imaging system. In the formula, when x, λ, and d are determined quantitatively, the contrast threshold and the Rayleigh criterion may solve for numerical aperture (opening diameter and angular opening) of the imaging system doing the observing. When that imaging system is the human eye, the opening diameter may be the iris (pupil size) of the patient. These quantities are optically related to the working F/# and foal length of the imaging system.

Once these parameters are quantified, a series of advanced optical raytrace analyses may be performed, resulting in the mathematical outputs of the key cardinal points, geometries, and metrics that define the performance of an optical system. When the optical system (as in the present application) is that of the human eye, the following cardinal points and geometries may be quantified: axial length (total length of eye from cornea to retina), refractive error sphere (myopia/nearsighted and hyperopia/farsighted defocus), refractive error cylinder (astigmatism), depth of focus, accommodation amplitude, spherical aberration, spherical equivalent power, focal point (back focal length of eye), presbyopia (nearpoint or front focal length of eye), and/or contrast sensitivity function (MTF). In some embodiments, AI/ML models may be used alongside the methods in order to optimize performance of the methods.

In some embodiments, the contrast acuity value derived for a patient may be related to key visual performance conditions that are attached to the contrast sensitivity function. As a result, each of the key visual performance conditions may be used in clinical refraction and diagnostic applications, such as: visual acuity screening, myopia management, detection of cataract formation, dry eye, macular degeneration, change in retina health, and presbyopia.

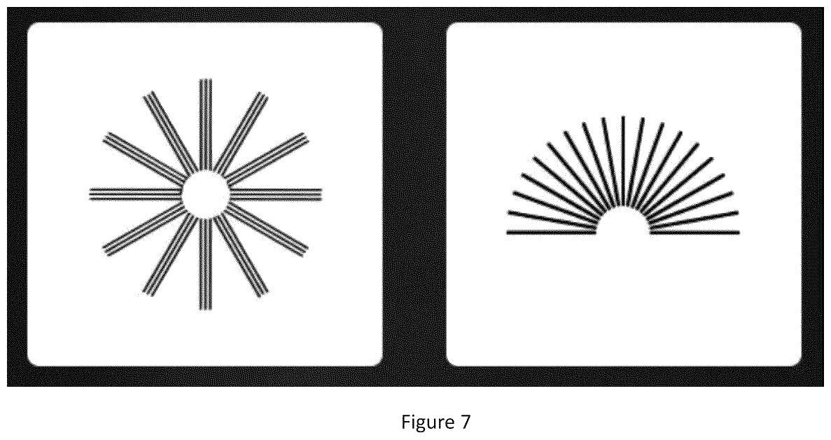

The system and platform may further the instruct the patient to move forward with an astigmatism test, presenting this test through a series of interfaces as part of the ongoing story. This may include, for example, interfaces asking the patient to perform a test including examining diagrams, symbols or images shown in user interfaces. Instructions are provided to the patient of the diagrams, symbols or images to be examined during the test. For example, interfaces may be provided with images such as those shown in FIG. 7. These may be shown on separate interfaces, and other images may also be shown that have a plurality of lines in the images. When the left eye is covered, the patient is asked to identify any lines or elements of the images, symbols or diagrams, to identify any differences (e.g. differences in color) and select them. When the right eye is covered, the patient is asked to do the same again, to identify any lines or elements of the images, symbols or diagrams for differences and select these differences. The user may make these identifications by clicking or touching lines, if the device includes a touchscreen. The user may also make identifications through a user input device such as a mouse.

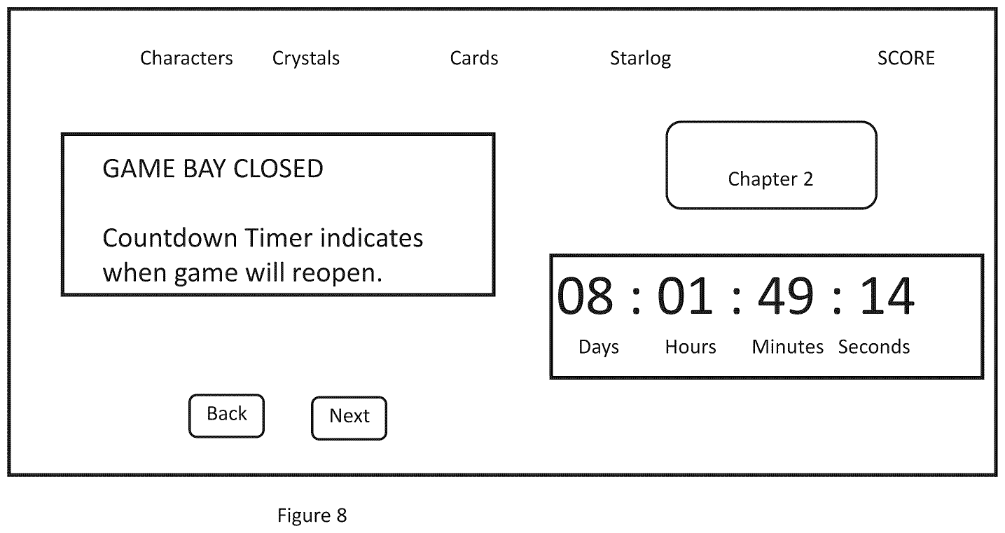

Conclusion of the test leads back to continuing with the story and the game (step 110 as shown in FIG. 1A). The patient continues to engage with the narration of the story, and participating in the game including for example, collecting crystals and competing to match the crystals with the characters of the story. For example, as shown at step 111 in FIG. 1A, the story and game concludes and the patient is shown an interface with a countdown timer, such as illustrated in FIG. 8, indicating the amount of time until they can access the eye test and the story at a future time (e.g. in one month). The test may be accessed by the administrator or parent and patient on a periodic basis including on a monthly basis through a notification email. As shown at step 112, an administrator of the account may further able to access an administrator page and review testing details and user information. A new chapter of the story may be added each time the platform is accessed, generally each month allowing the overall story to continue over time as the users participate with the platform and eye testing.

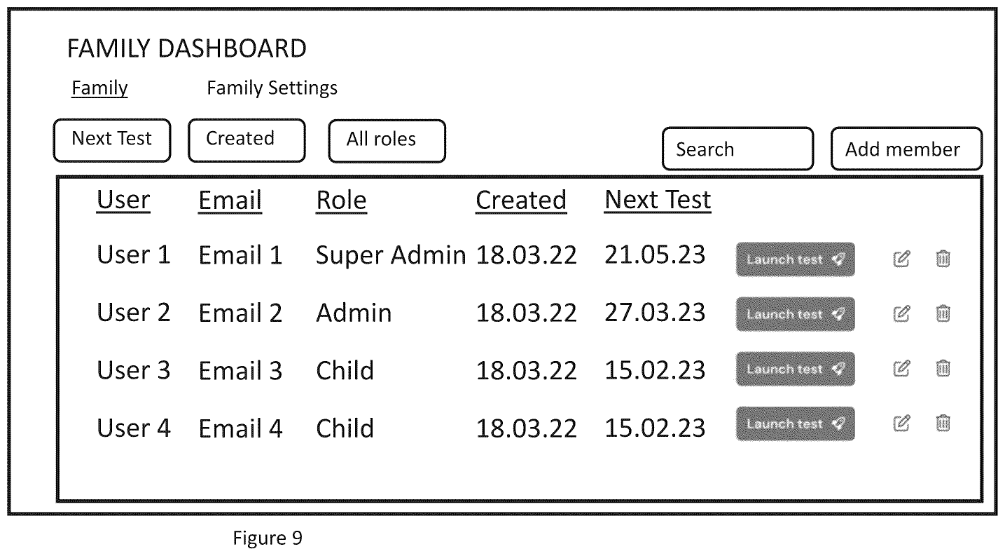

FIG. 9 depicts a dashboard display of account information with reference to the parent or administrator and/or the patient. At the end of the test and the story, the administrator or parent is lead back to the account or dashboard page where they can manage the profiles of both the administrator and patient (step 112). The portal further serves to provide access to eye health information, communicate with eye care professionals, amongst other options.

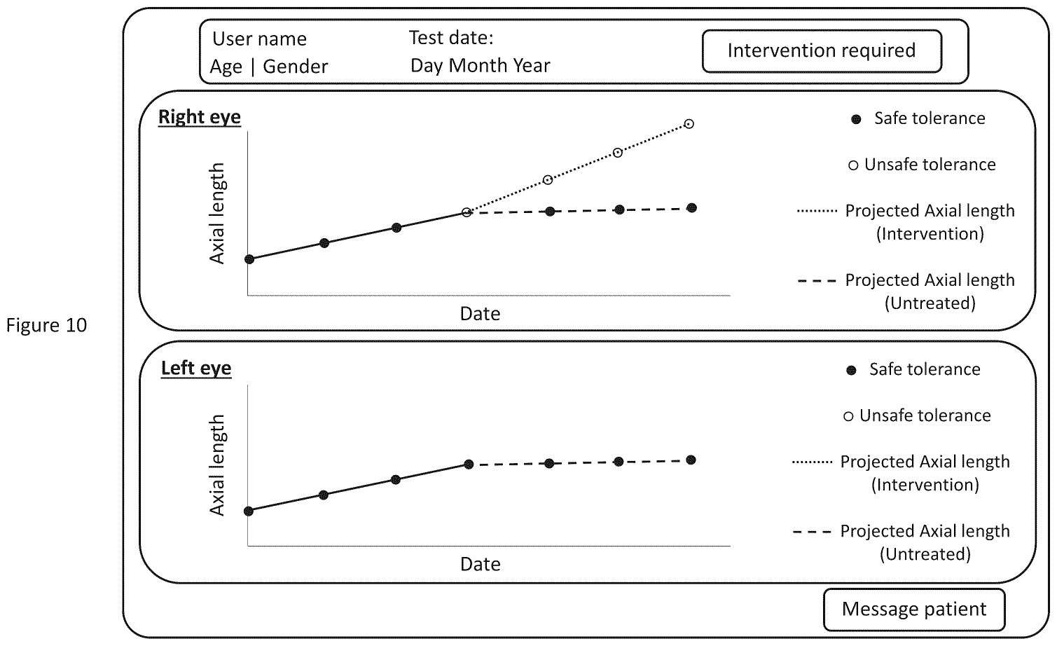

FIG. 10 shows an eye care professional or provider portal which displaying tracking information on eye care data, for example, axial length. The test data is provided on a periodic basis after each test is taken (e.g. monthly). This interface may displaying a portal to a provider, the portal including the eye data and at least two values associated with an axial length data associated with the first user, wherein the two values are associated with two separate points in time. Based on a difference between the at least two values associated with the axial length data and a threshold tolerance level, the system may a link to at least the first user to alert them of a further action required, such as an in-person visit.

The methods and systems described herein may be implemented using a computer program product. The computer program product can include one or more non-transitory, tangible, computer readable storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present disclosure.

Although the technology herein has been described with reference to particular embodiments, it is to be understood that these embodiments are merely illustrative of the principles and applications of the present technology. It is therefore to be understood that numerous modifications may be made to the illustrative embodiments and that other arrangements may be devised without departing from the spirit and scope of the present technology as defined by the appended claims.

1. A method for managing a story-led monthly eye test that monitors eye diseases, the method comprising:
receiving a link at a client device, from an eye care specialist in response to a diagnosis of the eye disease for tracking the progression of the eye disease;
displaying a prompt in the client device for a user to login or sign up to access the story-led monthly eye test;
login using an email or social media data associated with the user to access a platform providing the story-led monthly eye test;
receive input associated with user information;
sending the results to the eye care specialist;
generating on a platform operated by the eye care specialist, the test results;
displaying an alert, based on the test results exceeding a threshold; and
in response to the alert, sending a request from the eye care specialist to schedule video or in-person appointment.

2. A method for testing user for an eye disease, the method comprising:
receiving input from an administrator, based in part on a prompt for user information;
in response to the user information, determine the user qualifies to take the test;
select a screen size to display at least one image;
launch a game used for testing the eye disease;
accept a voice generated input from the user;
capture an image of the user to measure a pupillary distance;
measure the distance between the user and a client device; and
providing, based on the measured distance, a visual indication on a display of the client device.

3. A method for measuring eye data on a client device, the method comprising:
displaying at least three symbols on a display of the client device;
based on input from a user, decrease a size of at least one symbol of the at least three symbols on the display of the client device;
displaying at least one image associated with at least one color value;
in response to the user identifying the at least one image, modifying the at least the one color value associated with the image;
displaying a story associated with measuring the eye data;
generating a timer indicating when the user can access the test and the story;
in response to the timer, providing a prompt with link corresponding to the story and test; and
in response to selecting the link, accessing a new chapter of the story along with the test.

4. A method for managing eye data, the method comprising:
generating a story displayed on a client device to monitor the eye data associated with the first user;
displaying account information including at least two profiles associated with the first user and a second user;
accessing, based on input provided by the second user, eye data associated with the first user;
displaying a portal to a provider, the portal including the eye data and at least two values associated with an axial length data associated with the first user, wherein the two values are associated with two separate points in time; and
based on a difference between the at least two values associated with the axial length data and a threshold tolerance level, sending a link to at least the first user.

5. A method for measuring visual function, the method comprising:
displaying at least one image associated with at least one color value and a position on a display of a client device;
in response to a user identifying the at least one image, modifying the at least the one color value and the position of the at least one image;
generating a second image with the modified color value at the modified position;
receiving an input from a user associated with the at least second image;
determining, based on in part on the input from the user, a contrast threshold;
determining, based in part on a visual acuity model and the contrast threshold, at least one value associated with a contrast acuity; and
returning to a display of a narration in progress, the narration associated with measuring visual function.

6. A method for measuring eye data, the method comprising:
receiving user input including login data or social media information;
displaying account information including at least two profiles associated with the first user and a second user;
launching a game, response to input received from the first user;
accessing, based on input provided by the second user, eye data associated with the first user;
displaying a portal to a provider, the portal including the eye data and at least two values associated with an axial length data associated with the first user, wherein the two values are associated with two separate points in time; and
generating an alert on the portal, based on the two values associated with an axial length data and a threshold value.
